(19) United States
(12) Patent Application Publication
(10) Pub. No.: US 2024/0325484 A1
(43) Pub. Date: Oct. 3, 2024

(54) COMPOSITION COMPRISING HORSE CHESTNUT EXTRACT

(71) Applicant: ANGIOLAB, INC., Daejeon (KR)

(72) Inventors: Min Young KIM, Daejeon (KR); Byung Young PARK, Daejeon (KR); II Han CHO, Seoul (KR); Eun Kyu PARK, Gyeonggi-do (KR)

(21) Appl. No.: 18/580,366

(22) PCT Filed: Aug. 31, 2022
(86) PCT No.: PCT/KR2022/013018

§ 371 Date: Jan. 18, 2024

(30) Foreign Application Priority Data
Sep. 1, 2021 (KR) 10-2021-0116512

(51) Int. Cl. A61K 36/77; A61K 31/352; A61K 31/7048; A61P 1/02

(52) U.S. Cl. CPC A61K 36/77; A61K 31/352; A61K 31/7048; A61P 1/02; A61K 2236/333; A61K 2236/51

(57) ABSTRACT

The present invention relates to a pharmaceutical composition for preventing or treating periodontal disease, comprising horse chestnut extract as effective component, and a food composition for preventing or improving periodontal disease, comprising horse chestnut extract as effective component, wherein the horse chestnut extract comprises avicularin and juglanin as active ingredients.

### TECHNICAL FIELD

The present invention relates to a pharmaceutical composition for preventing or treating periodontal disease comprising horse chestnut extract as effective component, and a food composition for preventing or improving periodontal disease comprising horse chestnut extract as effective component.

### BACKGROUND ART

Periodontitis is a type of inflammation that occurs in supporting tissues around teeth by toxins, which are metabolites of microorganisms living in the oral cavity. This periodontal disease starts from gingivitis in the early stages develops into periodontitis accompanied by swelling of the gums, bleeding, and severe bad breath if left unattended. In addition, continuously progressing periodontitis develops into progressive periodontal disease in which collagen supporting the periodontal membrane is destroyed and the alveolar bone supporting the teeth is dissolved, the periodontal ligaments are separated to form a periodontal pocket, and in severe cases damage the teeth.

The etiology of periodontal disease varies depending on gender, race, and age. These periodontal diseases continue to worsen and lull over the years, and the infection rate is high in patients with systemic diseases such as diabetes, AIDS, neutropenia, and Down syndrome.

The most effective way to prevent periodontal disease can be achieved by using a substance that inhibits the activity of these microorganisms and facilitates blood flow to the inflamed gums. However, among the drugs used for the prevention and/or treatment of periodontal disease, antibiotics such as penicillin, erythromycin, and tetracycline are effective in sterilization and growth inhibition, but have large side effects such as causing the appearance of resistant bacteria, and antibacterial agents such as chlorhexidine have an effect of inhibiting the formation of caries, but has a problem of strong toxicity. Therefore, it is necessary to study natural products with fewer side effects.

In addition, with a growing use of a dental implant, various diseases associated with the implant have occurred a lot.

Out of those diseases, peri-implantitis is a disease that appears around the implant rather than a natural tooth, and thus requires most attentions after the implant. It is reported that peri-implantitis occurs from 60% or more of the implants ever performed. In particular, the implant has a problem in that it does not have nerves distributed therein, unlike the natural tooth, and thus an outbreak of peri-implantitis may not be well recognized in an early stage of development, but recognized only after serious worsening of symptoms.

However, if peri-implantitis is left untreated in its early stage of development, peri-implantitis may even cause an alveolar bone to collapse in a severe case. Thus, peri-implantitis requires a careful treatment, and a serious bone loss around the implant may go so far as to require a re-operation due to a failure of the implant.

Also, peri-implantitis progresses fast in comparison with other diseases developed in the natural tooth, and also tends to show a higher degree of severity.

However, the existing drugs used to prevent or treat peri-implantitis have not effectively or fundamentally treated peri-implantitis, but have simply alleviated its symptoms only. Thus, upon the occurrence of peri-implantitis, it has been difficult to treat peri-implantitis with a drug only, but other methods such as a surgical procedure, an operation or the like have been mainly used instead.

Thus, there is a need to develop a drug that may effectively prevent or treat peri-implantitis.

### PRIOR ART REFERENCES

### Patent Documents

(Patent Document 1) Korean Registered Patent Publication No. 10-2260037

### DISCLOSURE OF INVENTION

### Technical Problem

The present invention provides a pharmaceutical composition for preventing or treating periodontal diseases and a food composition for preventing or improving periodontal diseases which has no side effects for long-term administration, is safe due to having low cytotoxicity, and suppresses periodontal disease by inhibiting the differentiation of osteoclasts.

### Solution to Problem

The present inventors have recognized the need for research on the prevention or treatment of periodontal disease, and tried to achieve the purpose of research on natural substances capable of preventing or treating such diseases.

Specifically, the present inventors completed the present invention while studying natural products with no toxicity and no side effect even when administered for a long period of time, that can inhibit the differentiation of osteoclasts effectively to reduce bone loss to prevent/treat the onset of periodontal disease induced or caused by bone loss. It was confirmed that horse chestnut extract has the preventive and/or therapeutic effects on periodontal disease by inhibiting tartrate-resistant acid phosphatase (hereinafter referred to as ‘TRAP’).

Hereinafter, the present invention will be described in detail.

### Pharmaceutical Composition for Preventing or Treating Periodontal Disease Comprising Horse Chestnut Extract as Effective Component

The present invention is a pharmaceutical composition comprising horse chestnut extract as effective component for preventing or treating periodontal disease, and it provides a pharmaceutical composition comprising avicularin and juglanin as active ingredients of horse chestnut extract for preventing or treating periodontal disease.

According to the present invention, the term “Horse Chestnut” belongs to a family Hippocastanaceae, and is also called a Conker Tree as well as Marronier in France.

According to the exemplary embodiments of the present invention, horse chestnut extract may be obtained from leaves or seeds (Saraja), particularly from leaves.

For example, the horse chestnut extract according to the present invention may be used in such a way that horse chestnut leaves commercially available or leaves harvested from spring through to autumn are dried in the shade. Or the horse chestnut extract may be a dry extract of the horse chestnut, which is prepared from a horse chestnut seed, and the one that comprises triterpene glycosides, i.e. anhydrous aescin, in an amount of 10 wt % or more may be used. More preferably, an extract of horse chestnut leaves may be used.

According to the exemplary embodiments of the present invention, the horse chestnut may be Japanese horse chestnut (Aesculus turbinata BLUME), Chinese horse chestnut (Aesculus chinensis Bge and Aesculus wilculus Rehd), European horse chestnut (Aesculus hippocastanum L.) and the like, particularly the European horse chestnut (Aesculus hippocastanum L. Kalisz, Poland), but is not limited thereto.

According to the present invention, the term “extract” has the meaning commonly used as a crude extract in the art, but in a broad sense also includes a fraction obtained by additional fractionation of the extract, and specifically, the present invention includes the extract obtained by extraction, diluents or concentrates of the extract, dried products obtained by drying the extract, crude or purified or mixed products of the extract, extract of all formulations that can be formed using the extract and the extract itself.

According to the present invention, the term “effective component” refers to a substance or group of substances (including herbal medicines and the like for which pharmacologically active ingredients are not identified) expected to directly or indirectly express the efficacy of the composition by its intrinsic pharmacological action, which means to include the main component.

According to the present invention, the term “periodontal disease” refers to a disease that appears in tissues around teeth, such as the gingiva surrounding the teeth, the periodontal ligament, and the alveolar bone.

According to the present invention, the term “prevention” refers to any action of suppressing or delaying periodontal disease by inhibiting TRAP, which is an osteoclast differentiation marker, by administration of horse chestnut extract according to the present invention.

According to the present invention, the term “treatment” refers to any action for improving or beneficially changing periodontal disease by inhibiting TRAP, an osteoclast differentiation marker, by administration of horse chestnut extract according to the present invention.

According to the present invention, the horse chestnut extract comprises avicularin and juglanin as active ingredients.

Avicularin, an active ingredient in horse chestnut extract according to the present invention, may be represented by a following chemical formula 1.

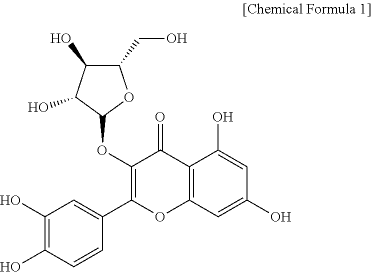

Juglanin, an active ingredient in horse chestnut extract according to the present invention, may be represented by a following chemical formula 2.

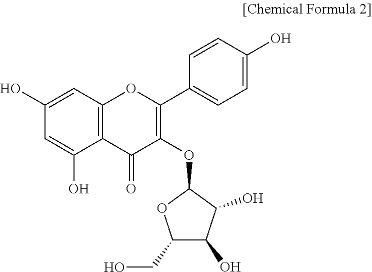 [SMILES: O=c1c(O[C@@H]2O[C@@H](CO)[C@H](O)[C@H]2O)c(-c2ccc(O)cc2)oc2cc(O)cc(O)c12]

According to the present invention, the horse chestnut extract may comprise avicularin as an active ingredient, in an amount of 0.1 to 4.0 wt %, particularly in an amount of 0.3 to 3.0 wt %, more particularly 0.5 to 2.0 wt % based on the total weight of horse chestnut extract.

According to the present invention, the horse chestnut extract may comprise juglanin as an active ingredient, in an amount of 0.1 to 3.0 wt %, particularly in an amount of 0.12 to 2.0 wt %, more particularly 0.15 to 1.0 wt % based on the total weight of horse chestnut extract.

According to the present invention, the horse chestnut extract may further comprise quercitrin and afzelin as another active ingredients.

Quercitrin, an active ingredient in horse chestnut extract according to the present invention, may be represented by a following chemical formula 3.

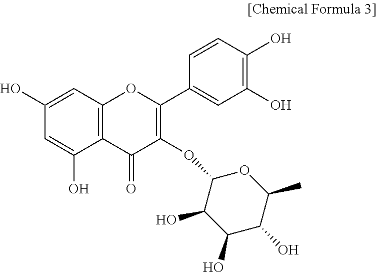 [SMILES: C[C@@H]1O[C@@H](Oc2c(-c3ccc(O)c(O)c3)oc3cc(O)cc(O)c3c2=O)[C@H](O)[C@H](O)[C@H]1O]

Afzelin, an active ingredient in horse chestnut extract according to the present invention, may be represented by a following chemical formula 4.

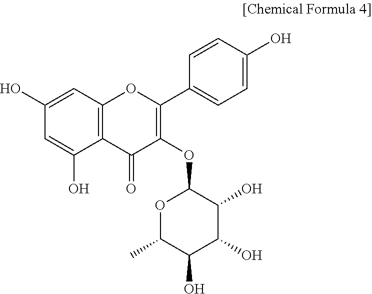 [SMILES: C[C@@H]1O[C@@H](Oc2c(-c3ccc(O)cc3)oc3cc(O)cc(O)c3c2=O)[C@H](O)[C@H](O)[C@H]1O]

According to the present invention, the horse chestnut extract may comprise quercitrin as an active ingredient, in an amount of 1.0 to 7.0 wt %, particularly in an amount of 1.3 to 6.0 wt %, more particularly 1.5 to 5.0 wt % based on the total weight of horse chestnut extract.

According to the present invention, the horse chestnut extract may comprise afzelin as an active ingredient, in an amount of 0.1 to 3.0 wt %, particularly in an amount of 0.2 to 2.0 wt %, more particularly 0.3 to 1.0 wt % based on the total weight of horse chestnut extract.

That is, the horse chestnut extract may comprise in an amount of 0.1 to 4.0 wt % of avicularin, 1.0 to 7.0 wt % of quercitrin, 0.1 to 3.0 wt % of juglanin, and 0.1 to 3.0 wt % of afzelin, particularly (1) 0.3 to 3.0 wt % of avicularin, 1.3 to 6.0 wt % of quercitrin, 0.12 to 2.0 wt % of juglanin, and 0.2 to 2.0 wt % of afzelin (2) 0.5 to 2.0 wt % of avicularin, 1.5 to 5.0 wt % of quercitrin, 0.15 to 1.0 wt % of juglanin, and 0.3 to 1.0 wt % of afzelin as active ingredients based on the total weight of horse chestnut extract.

Hereinafter, in the exemplary embodiments, horse chestnut extract of the present invention may comprise avicularin, quercitrin, juglanin and afzelin in the above-described weight %, unless otherwise specified.

According to the present invention, the contents of avicularin, quercitrin, juglanin and afzelin in horse chestnut extract can be measured by quantifying the contents of the avicularin, quercitrin, juglanin and afzelin comprised in the extract.

Since avicularin, quercitrin, juglanin, and afzelin comprised in horse chestnut extract of the present invention may be converted into other substances during the quantification process, the quantification process needs to be strictly controlled in order to accurately quantify the components in horse chestnut extract.

In particular, when horse chestnut extract of the present invention comprises the avicularin and the juglanin in a weight ratio within the above range, the prevention or treatment effect of periodontal disease may be excellent.

Specifically, avicularin and juglanin as active ingredients in the horse chestnut extract are new components that have not been disclosed as active ingredients in horse chestnut extract, and avicularin and juglanin can be considered functional components to inhibit the differentiation of osteoclasts.

However, the content of avicularin and juglanin in the horse chestnut extract may change within the range in which the efficacy is maintained according to the region, year, and harvest time.

According to the present invention, the pharmaceutical composition has an effect on the prevention and treatment of periodontal disease by inhibiting TRAP, an osteoclast differentiation marker, and has excellent pharmacological effects with fewer side effects.

Specifically, in bone metabolism and bone formation, bone homeostasis is maintained by a balance between bone formation by osteoblasts and bone resorption by osteoclasts. Osteoclasts adhere to the surface of bone and secrete acid and proteolytic enzymes to remove the bone matrix constituting the bone and dissolve the aged bone.

When osteoclast differentiation is activated, various enzymes to degrade bone are produced. TRAP, an acidic phosphatase enzyme that oxidizes and degrades bone, is used as a special marker for osteoclasts.

Specifically, the secretion of TRAP increases when osteoclasts perform bone resorption, and the activity is high when ATP and nitrophenyl phosphate are present. TRAP is a cytochemical marker enzyme that can measure the degree of differentiation of osteoclasts.

Therefore, the method for evaluating the inhibitory effect on osteoclast differentiation is to analyse the activity of TRAP, and it can be confirmed that the pharmaceutical composition according to the present invention can prevent bone destruction by inhibiting the activity of TRAP.

In another aspect, 0.5 to 2.0 g of avicularin may be comprised as an active ingredient based on 100 g of the horse chestnut extract.

In another aspect, 1.5 to 5.0 g of quercitrin may be comprised as an active ingredient based on 100 g of the horse chestnut extract.

In another aspect, 0.15 to 1.0 g of juglanin may be comprised as an active ingredient based on 100 g of the horse chestnut extract.

In another aspect, 0.3 to 1.0 g of afzelin may be comprised as an active ingredient based on 100 g of the horse chestnut extract.

According to the present invention, the horse chestnut extract may be obtained by an extraction process comprising extracting and concentrating horse chestnut leaves with 10% to 50% (v/v) of aqueous alcohol solution.

According to the present invention, the term “alcohol” refers to a compound in which a hydroxyl group is bonded to a carbon atom of an alkyl or substituted alkyl, and the term “alkyl” refers to a linear saturated hydrocarbon group or branched saturated hydrocarbon group, and the term “substituted alkyl group” means that a substituent bonded to the carbon of the alkyl group is hydroxy, cyano, halides, etc.

According to the exemplary embodiments of the present invention, the aqueous alcohol may be 20-30% (v/v) of aqueous alcohol solution.

The aqueous alcohol solution refers to a C1-C4 alcohol including 20-30% (v/v) aqueous ethanol solution, an aqueous methanol solution, etc., preferably an aqueous ethanol solution or an aqueous methanol solution, more preferably 25% (v/v) aqueous ethanol solution.

According to the present invention, the term ‘C1-C4’ means a functional group or main chain having 1 to 4 carbon atoms.

According to the present invention, the extract may be dried using a hot air drying method, or a freeze drying method, or a spray drying method. For example, the extract may be hot-air dried using a hot-air dryer, or freeze-dried by freezing the extract and lowering the air pressure, or spray-dried by spraying the extract and blowing hot air.

In the preparation of horse chestnut extract of the present invention, dried or undried horse chestnut leaves or a mixture thereof may be used. For effective extraction, horse chestnut leaves can be finely crushed and used.

According to the present invention, the alcohol extract may be obtained by the following steps, but is not limited thereto.

(S1) An extraction step to obtain an alcohol extract by extracting horse chestnut leaves with 10% to 50% (v/v) aqueous alcohol solution at 75 to 85° C. for 1 to 5 hours;

(S2) a step to obtain a concentrated extract by concentrating the alcohol extract under a temperature condition of 25 to 35° C., and a pressure condition of −755 to −650 mmHg and;

(S3) a step to obtain horse chestnut extract by adding purified water to concentrated extract to have 20˜40% Brix concentrations, dissolving it and then freeze-drying the dissolved concentrated extract.

According to the present invention, the alcohol extract in (S1) may be prepared with 10 ˜50% (v/v) aqueous alcohol solution of 5 to 15 times (v/w) of the horse chestnut leaves, preferably 20˜30% (v/v) aqueous alcohol solution, and step (S1) can be repeated 1, 2 or 3 times, for example, when repeating 2 or 3 times, the alcohol extract of each step is obtained, separated and finally mixed to obtain an alcohol extract.

The pharmaceutical composition for preventing or treating periodontal disease may comprise the horse chestnut extract as effective component in an amount of 10 to 90% by weight based on the total weight. Particularly the horse chestnut extract in an amount of 15 to 70% by weight, more particularly the horse chestnut extract in an amount of 40 to 60% by weight, and even more particularly the horse chestnut extract in an amount of 45 to 55% by weight may be comprised.

According to the present invention, the periodontal disease may be one or more selected from the group consisting of gingivitis, periodontitis, alveolar pyorrhea, and peri-implantitis.

According to the present invention, the term “gingivitis” refers to inflammation of the gingiva, in other words, the gums. It can be classified into simple gingivitis, ulcerative gingivitis, gangrene gingivitis, and hypertrophic gingivitis.

According to the present invention, the term “periodontitis” is also called paradentitis, and refers to inflammatory diseases affecting periodontal tissue.

According to the present invention, the term “alveolar pyorrhea” refers to chronic marginal periodontitis and is a symptom of purulent pericementitis.

According to the present invention, the term “peri-implantitis” refers to an inflammatory disease occurring in the gingival bone tissue around dental implant.

The pharmaceutical composition of the present invention for preventing or treating periodontal disease comprising horse chestnut extract comprises horse chestnut extract as effective component, and may further comprise a pharmaceutically acceptable carrier to be formulated in oral dosage form such as powder, granule, tablet, capsule, suspension, emulsion, syrup, aerosol, and for external use and sterile injection solution according to a conventional method.

According to the present invention, the term “pharmaceutically acceptable” refers to a composition that is physiologically acceptable and does not normally cause allergic reactions such as gastrointestinal disorders, dizziness, or similar reactions when administered to humans.

According to the present invention, the term “pharmaceutically acceptable carrier” typically includes a liquid or non-liquid basis of a pharmaceutical composition. If a pharmaceutical composition is provided in liquid form, the carrier typically includes pyrogen-free water; Isotonic saline or buffered (aqueous) solutions, for example phosphate, citric acid, etc. The injection buffer may be hypertonic, isotonic or hypotonic in a particular reference medium, i.e. the buffer may have a high, equal or low salt content in the particular reference medium, preferably, such concentrations of the aforementioned salts, which do not induce cell damage by osmotic pressure or other concentration effect, may be used.

The reference medium occurs in an “in vivo” method, for example, such as blood, lymph, cytoplasmic liquid, or other bodily fluid, or a bodily fluid that can be used as a reference medium in an “ex vivo” method, for example as a general buffer or liquid. Such general buffers or liquids are known to a person skilled in the art.

The pharmaceutically acceptable carrier may include those commonly used in the art for example, lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate and mineral oil or the like, but is not limited thereto.

In addition, the pharmaceutical composition of the present invention may comprise a diluent or excipient such as a filler, an extender, a binder, a humectant, a disintegrant, a surfactant, and other pharmaceutically acceptable additives.

The pharmaceutical composition of the present invention may be manufactured in the form of liquid, suspension, powder, granule, tablet, capsule, pill or extract.

The composition of the present invention may be administered orally or parenterally (e.g. liniments or intravenous, subcutaneous, intraperitoneal injection).

According to the present invention, the term “oral administration” is a method of injecting a drug by mouth for alleviating pathological symptoms (periodontal disease), and according to the present invention, the term “parenteral administration” refers to a method of subcutaneous, intramuscular, intravenous, or intraperitoneal administration using a tube, except for administration by mouth.

Solid dosage forms for oral administration may include powder, granule, tablet, capsule, soft capsule, pill and the like. Liquid dosage forms for oral administration may include suspension, liquid for internal use, emulsion, syrup, aerosol, and the like, and may include various additives, for example, humectant, sweetening agent, flavoring agent, preservative and the like in addition to water and liquid paraffin, which are frequently used simple diluents.

For dosage forms of parenteral administration, external preparations such as sterilized aqueous solution, liquid preparation, non-aqueous solvent, suspending agent, emulsion, eye drop, eye ointment, syrup, suppository, aerosol, etc., and sterile injection preparation can be formulated and used according to the conventional method, preferably cream, gel, patch, spray, ointment, plaster, lotion, liniment, eye ointment, eye drop, pasta or cataplasma pharmaceutical preparation can be formulated, but not limited thereto. Compositions for topical administration may be anhydrous or aqueous, depending on the clinical prescription. As non-aqueous solvent and suspending agent, propylene glycol, polyethylene glycol, vegetable oils such as olive oil, and injectable esters such as ethyl oleate may be used. As a base of suppository, witepsol, macrogol, tween 61, cacao butter, laurin, glycerogelatin, and the like may be used.

The pharmaceutically acceptable additive according to the present invention may be included in 0.1 to 99.9 parts by weight, specifically 0.1 to 50 parts by weight, based on the composition, but is not limited thereto.

According to the present invention, the pharmaceutical composition for preventing or treating periodontal disease comprising horse chestnut extract as effective component showed an inhibitory effect on TRAP, thereby having the effect of preventing or treating periodontal disease. The pharmaceutical composition for preventing or treating periodontal disease comprising horse chestnut extract as effective component can be administered in the conventional way by oral, rectal, intravenous, intraarterial, intraperitoneal, intramuscular, intrasternal, transdermal, topical, intraocular or intradermal routes and specifically by oral route.

The pharmaceutical composition for preventing or treating periodontal disease comprising horse chestnut extract as effective component of the present invention is preferably administered at a dose of 0.001 mg/kg to 50 mg/kg when administered once or several times a day.

### Food Composition Comprising Horse Chestnut Extract as Effective Component

It is to provide a food composition comprising horse chestnut extract as effective component, which comprises avicularin and juglanin as active ingredients for preventing or improving periodontal disease.

According to the present invention, horse chestnut extract comprises 0.6 to 1.5 wt. % of avicularin and 0.2 to 0.4 wt. % of juglanin as active ingredients based on the total weight of horse chestnut extract.

The horse chestnut extract and the preparation method thereof are the same as described above.

The food composition is for improving or preventing periodontal disease.

According to the present invention, the term “improvement” means any action that at least reduces the severity of symptoms of periodontal disease.

In the food composition according to the present invention, when horse chestnut extract is used as an additive in the food composition, it can be added as it is or used with other foods or food ingredients, and can be used appropriately according to a conventional method. The mixing amount of the effective components may be appropriately determined according to each purpose of use, such as prevention, health or treatment.

The formulation of the food composition according to the present invention may be possible in any form of general food or beverage as well as powder, granule, pill, tablet, and capsule.

There is no particular limitation on the type of the food, and examples of the food to which the substance can be added include meat, sausage, bread, chocolate, candy, snacks, confectionery, pizza, ramen, other noodles, gums, and dairy products including ice cream, various soups, beverages, teas, alcoholic beverages, and vitamin complex, and may include all foods in a conventional sense.

In general, in the manufacture of food or beverage, the horse chestnut extract may be added in an amount of 15 parts by weight or less, preferably 10 parts by weight or less, based on 100 parts by weight of the raw material. However, in the case of long-term intake for health and hygiene or health control, the amount may be less than the above range. In addition, since there is no problem in terms of safety using an extract from a natural product in the present invention it can also be used in an amount above the range.

Beverages in the food composition according to the present invention, may comprise various flavoring agents or natural carbohydrates as additional ingredients like conventional beverages. The natural carbohydrates may be monosaccharides such as glucose and fructose, disaccharides such as maltose and sucrose, polysaccharides such as dextrin and cyclodextrin, and sugar alcohols such as xylitol, sorbitol, and erythritol. As a sweetener, natural sweeteners such as thaumatin and stevia extract, or synthetic sweeteners such as saccharin and aspartame may be used. The ratio of the natural carbohydrate may be about 0.01 to 0.04 g, preferably about 0.02 to 0.03 g per 100 mL of the beverage according to the present invention.

In addition to the above, the food composition according to the present invention includes various nutrients, vitamins, electrolytes, flavoring agents, coloring agents, pectic acid and its salts, alginic acid and its salts, organic acids, protective colloidal thickeners, pH adjusters, stabilizers, preservatives, glycerin, alcohol, and carbonating agents used in carbonated beverages. In addition, the food composition of the present invention may comprise natural fruit juice, and fruit pulp for making fruit juice and vegetable beverage. These components may be used independently or in combination. The ratio of these additives is not limited, but is generally selected in the range of 0.01 to 0.1 parts by weight relative to 100 parts by weight of the food composition of the present invention.

### Method for Prevention or Treatment

Another object of the present invention is to provide a method for preventing or treating periodontal disease by administering a therapeutically effective amount of pharmaceutical composition comprising horse chestnut extract as effective component to a subject in need of treatment.

According to the embodiments of the present invention, the term “subject in need of treatment” refers to mammals including humans, and the term “administration” refers to providing a predetermined substance to a patient by any suitable method. In the treatment method of the present invention, the pharmaceutical composition for preventing or treating periodontal disease comprising horse chestnut extract as effective component may be administered in a conventional manner by oral, rectal, intravenous, intraarterial, intraperitoneal, intramuscular, intrasternal, transdermal, topical, intraocular or intradermal route.

In the method for preventing or treating periodontal disease of the present invention, horse chestnut extract of the present invention may be administered in a conventional manner by oral, rectal, intravenous, intraarterial, intraperitoneal, intramuscular, intrasternal, transdermal, topical, intraocular or intradermal routes, and specifically, it can be administered orally.

According to the embodiments of the present invention, the term “therapeutically effective amount” refers to the amount of an effective component or a pharmaceutical composition that induces a biological or medical response in a tissue system, animal, or human that is considered by researchers, veterinarians, medical doctors, or other clinicians, which includes an amount that induces amelioration of the symptoms of the disease or disorder being treated. It is apparent to those skilled in the art that the therapeutically effective dosage and frequency of administration of the effective component of the present invention will vary depending on the desired effect. Therefore, the optimal dosage of administration can be easily determined by those skilled in the art, and can be adjusted by various factors including the type of disease, the severity of the disease, the content of effective components and other ingredients comprised in the composition, the type of formulation, and patient's age, weight, general health, sex and diet, administration time, administration route and secretion rate of the composition, treatment period, and concomitant drugs.

In the method for preventing or treating periodontal disease of the present invention, horse chestnut extract of the present invention is preferably administered to adults at a dose of 0.001 mg/kg to 50 mg/kg when administered once or several times a day.

### Use of a Pharmaceutical Composition Comprising Horse Chestnut Extract as Effective Component for Preventing or Treating Periodontal Disease

An object of the present invention to provide the use of a pharmaceutical composition comprising horse chestnut extract as effective component for preventing or treating periodontal disease.

### Use of a Pharmaceutical Composition Comprising Horse Chestnut Extract as Effective Component for the Manufacture of a Medicament for Preventing or Treating Periodontal Disease

An object of the present invention is to provide the use of a pharmaceutical composition comprising horse chestnut extract as effective component for the manufacture of a medicament for preventing or treating periodontal disease.

According to the exemplary embodiments of the present invention, the pharmaceutical composition comprising horse chestnut extract as effective component for the manufacture of a medicament may be admixed with an acceptable carrier or the like, and may further comprise other agents.

### Other Daily Products Containing Pharmaceutical Composition for Preventing or Treating Periodontal Disease Comprising Horse Chestnut Extract as Effective Component

An object of the present invention is to provide toothpaste containing a pharmaceutical composition comprising horse chestnut extract as effective component for preventing or treating periodontal disease.

According to the present invention, when the pharmaceutical composition for preventing or treating periodontal disease is used as toothpaste, the composition may be added to toothpaste as it is, or mixed or added with a functional food or functional ingredient, and used appropriately according to a conventional method. The mixing amount of the effective components may be suitably determined according to the purpose of use.

Specifically, it may include additives commonly used in toothpaste including a fluorine compound, a wetting agent, a foaming agent, a binder, a sweetener, an abrasive agent, a flavoring agent, a preservative, and the like, and the specific type thereof is not limited as long as it can be used in toothpaste in general and in various ways.

Another object of the present invention is to provide a gargle solution comprising a pharmaceutical composition for preventing or treating periodontal disease.

The gargle solution includes a composition capable of gargling the oral cavity, a fluorine compound for cleaning and antibacterial activity in the oral cavity, a wetting agent, a foaming agent, a binder, a sweetener, an abrasive, a flavoring agent, a preservative agent, etc. and the specific type thereof is not limited as long as it can be used for general gargle liquid. The specific formulation is defined as including both the composition and mixing range that can be selected and mixed by a person skilled in the art.

Another object of the present invention is to provide an oral spray comprising a pharmaceutical composition for preventing or treating periodontal disease.

The oral spray may include all the composition and other agents for cleaning and antibacterial activity in the oral cavity. The specific formulation is defined as including all the composition and mixing range that can be selected and mixed by a person skilled in the art.

The pharmaceutical composition, treatment method, use, and other daily products of the present invention are equally applied unless they contradict each other.

Advantages and features of the present invention and methods for achieving them will become apparent with reference to the exemplary embodiments described below in detail. However, the present invention is not limited to the exemplary embodiments disclosed below, but will be implemented in a variety of different forms. These exemplary embodiments just make the disclosure of the present invention complete, and are provided to inform the scope of the invention completely to those of ordinary skill in the art to which the present invention pertains, and the present invention is only defined by the scope of the claims.

### Advantageous Effects of Invention

Horse chestnut extract of the present invention is safe with low cytotoxicity, and shows an inhibitory effect on osteoclast differentiation.

Therefore, the composition comprising horse chestnut extract of the present invention as effective component can be useful for the prevention or treatment of periodontal disease, has significantly fewer side effects, and has the effect of preventing or treating periodontal disease.

### BRIEF DESCRIPTION OF DRAWINGS

FIG. 1 is showing the result of high-performance liquid chromatography (hereinafter referred to as ‘HPLC’) of horse chestnut extract prepared according to Example 1 of the present invention.

FIG. 2 is showing the HPLC result of avicularin.

FIG. 3 is showing the HPLC result of quercitrin.

FIG. 4 is showing the HPLC result of juglanin.

FIG. 5 is showing the HPLC result of afzelin.

FIG. 6 is the result of a cytotoxicity assay of horse chestnut extract prepared according to Example 1 of the present invention.

FIG. 7 is the result of a cytotoxicity assay of avicularin

FIG. 8 is the result of a cytotoxicity assay of quercitrin.

FIG. 9 is the result of a cytotoxicity assay of juglanin.

FIG. 10 is the result of a cytotoxicity assay of afzelin.

FIG. 11 is showing the inhibition of TRAP activity by horse chestnut extract prepared according to Example 1 of the present invention.

FIG. 12 is showing the inhibition of TRAP activity by avicularin.

FIG. 13 is showing the inhibition of TRAP activity by quercitrin.

FIG. 14 is showing the inhibition of TRAP activity by juglanin.

FIG. 15 is showing the inhibition of TRAP activity by afzelin.

### BEST MODE FOR CARRYING OUT THE INVENTION

The terms used in this specification have been selected as currently widely used general terms as possible while considering the functions according to the present invention, but these may vary depending on the intention of a person skilled in the art or precedent, the emergence of new technology, and the like. In addition, in a specific case, there is a term arbitrarily selected by the applicant, and in this case the meaning will be described in detail in the description of the present invention. Therefore, the term used according to the present invention should be defined based on the meaning of the term and the overall content of the present invention, rather than the name of a simple term.

Unless otherwise defined, all terms used herein including technical or scientific terms have the same meaning as commonly understood by those skilled in the art to which the present invention pertains. Terms such as those defined in a commonly used dictionary shall be interpreted as having a meaning consistent with the meaning in the context of the related art, and shall not be interpreted in an ideal or excessively formal meaning unless explicitly defined in the present application.

The numerical ranges include the numbers defined in the range. All maximum numerical limits given throughout the present specification include all lower numerical limits as if the low numerical limits were expressly written. All minimum numerical limits given throughout the present specification include all higher numerical limits as if the high numerical limits were expressly written. All numerical limits given throughout this specification will include all numerical ranges that are better within the broader numerical range, as if the narrower numerical limits were expressly written.

Examples and manufacturing examples are presented to help the understanding of the present invention. The following examples and manufacturing examples are only provided for easier understanding of the present invention, and the content of the present invention is not limited by the examples and manufacturing examples.

### EXAMPLES

### Example 1. Preparation of Horse Chestnut Extract

The leaves of horse chestnut (Aesculus hippocastanum L.) produced in Kalisz, Poland, were dried at room temperature in the shaded and then crushed. 80 kg of crushed leaves were extracted with 800 L of 25% (v/v) aqueous ethanol solution at 80° C. for three hours and filtered through a 25 μm cartridge filter to obtain 450 L of the first extract.

The residue of horse chestnut leaves was extracted with 800 L of 25% (v/v) aqueous ethanol solution at 80° C. for 3 hours, and filtered through a 25 μm cartridge filter to obtain 830 L of the second extract.

After mixing the first and second extracts (a total of 1280 L of extract), it was concentrated at a temperature of 75-85° C. under the conditions of −755 to −650 mmHg to obtain 20 L of a concentrated extract.

After that, 50 L of purified water was added to the concentrated extract and concentrated under reduced pressure at 20-30° C. to remove the ethanol solvent, 60 L of purified water was added and dissolved to a concentration of 25-35% Brix. After freeze-drying in a lyophilizer, 18 kg of freeze-dried powder of horse chestnut extract was obtained by pulverizing at 1,200 rpm on a 0.5 mm pulverizing net in a pulverizer.

### Example 2. Ingredients of Horse Chestnut Extract

### (1) Analytical Method

It was confirmed as follows whether or not the horse chestnut extract comprises avicularin, quercitrin, juglanin and afzelin, particularly avicularin and juglanin which are not known to be comprised in horse chestnut extract as active ingredients.

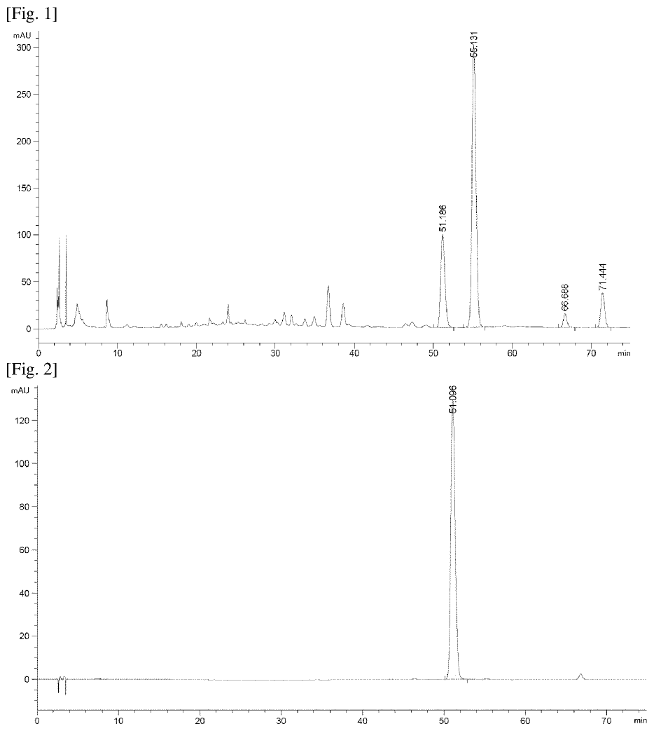

100 mg of horse chestnut extract was placed in a tube, sufficiently dissolved in 100 mL of 100% methanol, filtered through a 0.22 μm filter, and HPLC was performed. The result was shown in FIG. 1.

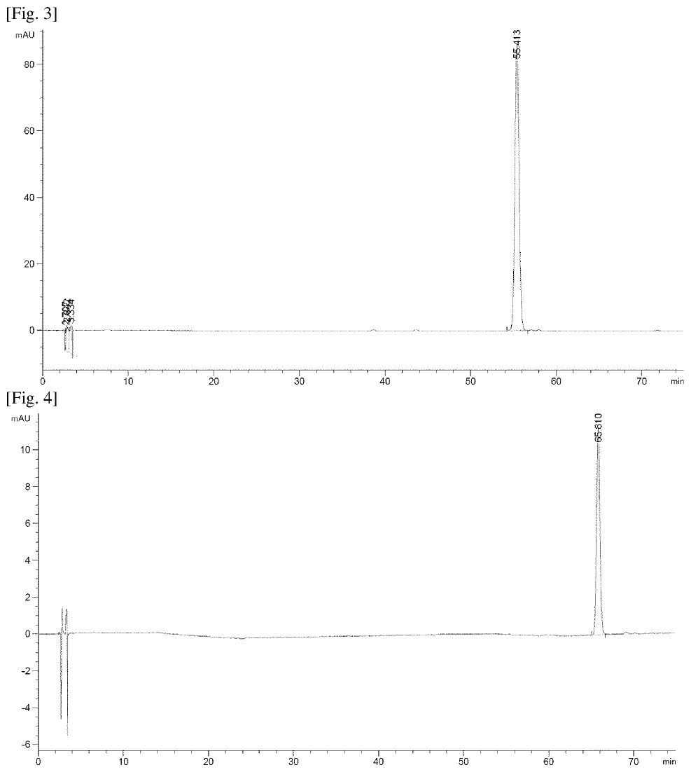

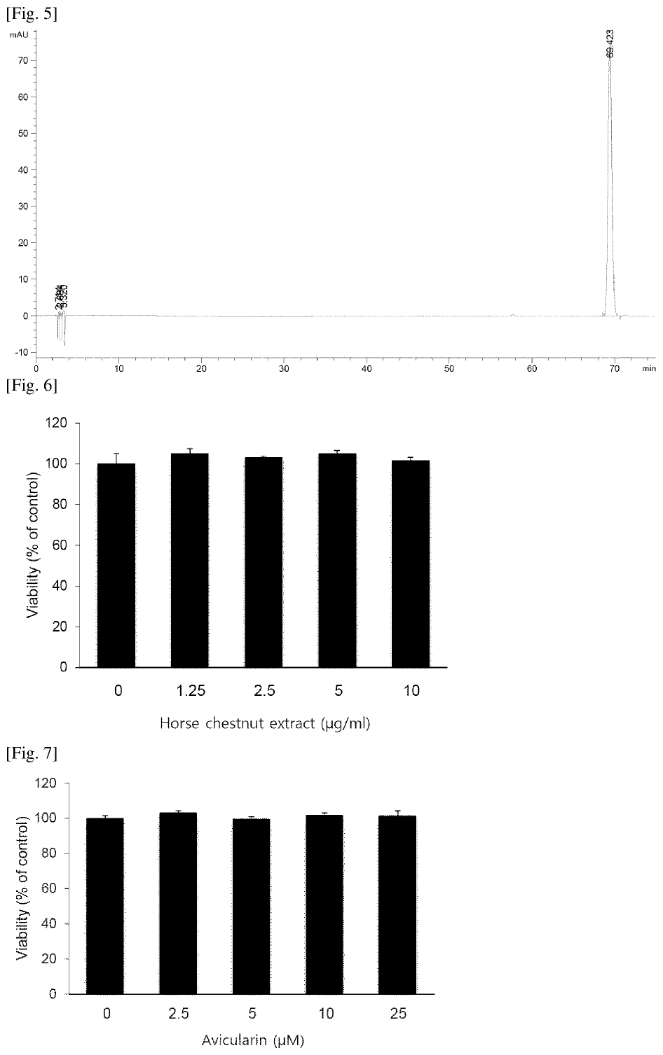

In addition, HPLC was performed on each of avicularin, quercitrin, juglanin, and afzelin, and the results were shown in FIGS. 2 to 5, respectively.

The conditions for performing HPLC were as follows.
i) Column: CAPCELL PAK C18 UG120 (SHISEIDO, 4.6×250 mm, 5 μm)
ii) Detector: UV 254 nm
iii) Flow rate: 1.0 mL/min
iv) Mobile phase

Mobile phase A: 0.1% (w/v) phosphoric acid, Mobile phase B: Acetonitrile

TABLE 1

| Time (min) | Mobile phase A | Mobile phase B |
|---|---|---|
| 0 | 90 | 10 |
| 10 | 90 | 10 |
| 20 | 83 | 17 |
| 50 | 83 | 17 |
| 55 | 80 | 20 |
| 75 | 80 | 20 |
| 76 | 0 | 100 |
| 90 | 0 | 100 |

### (2) Results

Horse chestnut extract obtained according to Example 1 comprised avicularin, quercitrin, juglanin, and afzelin according to the above analytical method, and in particular, it was confirmed that avicularin and juglanin which are not known as active ingredients of horse chestnut extract were comprised.

These contents were as follows.
Avicularin: 0.7% by weight
Quercitrin: 2.2% by weight
Juglanin: 0.2% by weight
Afzelin: 0.4% by weight

### Preparation of Reagents

Avicularin, quercitrin, juglanin, and afzelin were purchased from Sigma-Aldrich Korea, and other reagents were also purchased from Sigma-Aldrich, Sigma-Aldrich Korea and Duksan General Science.

### EXPERIMENTAL EXAMPLES

### Experimental Example 1. Cytotoxicity Assay

### (1) Experimental Method

A murine macrophage cell line, RAW264.7 cells were dispensed into a 96-well plate (Corning, USA) so that the number of cells was 1×10³ cells/well, and then cells were treated with horse chestnut extract of the present invention at concentrations of 1.25, 2.5, 5, and 10 (g/ml respectively and cultured at 37° C. for 4 days in 5% CO₂ incubator. In addition, avicularin, quercitrin, juglanin, and afzelin, which are the main components of horse chestnut extract, were treated at concentrations of 2.5, 5, 10, and 25 μM, respectively and cultured in the same manner. All 100 μl of the culture media was removed from each well, and after washing each well with 1×PBS (phosphate buffered saline), 100 μl of fresh medium was added. The cells were treated with 50 μl of XTT reagent per well, then reacted for four hours. An absorbance change thereof was measured at 490 nm and 650 nm (reference wavelength) by ELISA (enzyme linked immunosorbent assay) reader (Molecular Devices, USA), and a cell viability was expressed as a percentage.

### (2) Results

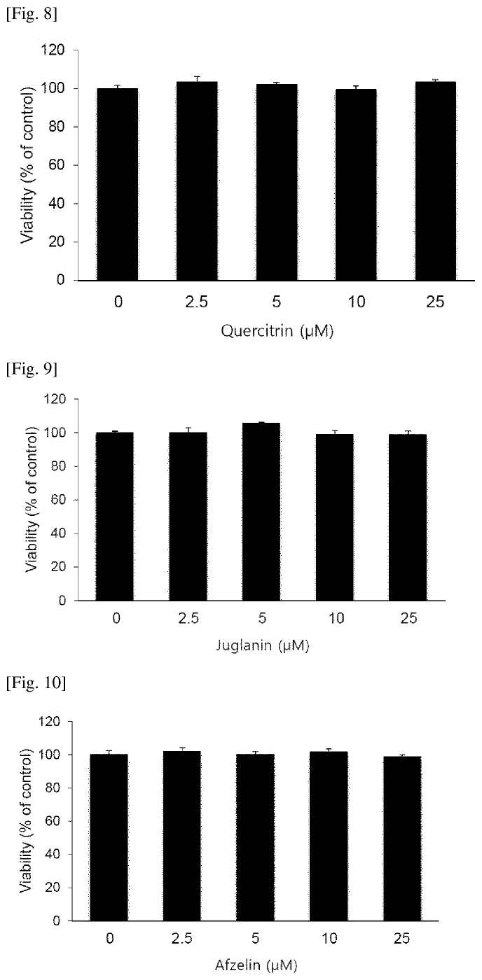

From the result of FIG. 6, it can be seen that horse chestnut extract according to Example 1 of the present invention had no cytotoxicity at concentrations of 25, 50, 75, and 100 μg/mL, and in addition, avicularin, quercitrin, juglanin, and afzelin had no cytotoxicity at concentrations of 2.5, 5, 10, and 25 μM respectively (see FIG. 7 to FIG. 10).

### Experimental Example 2, TRAP Inhibitory Activities of Horse Chestnut Extract and its Main Ingredients

### (1) Experimental Method

To confirm the effect of horse chestnut extract on osteoclast differentiation, a receptor activator of nuclear factor-kappa B ligand (hereinafter referred to as ‘RANKL’) was treated in a mouse macrophage cell line (hereinafter referred to as ‘Raw 264.7 cells’) to induce osteoclast differentiation. Horse chestnut extract according to Example 1 and the main ingredients of the horse chestnut extract, i.e. avicularin, quercitrin, juglanin, and afzelin were treated respectively and the activities of TRAP, an osteoclast-specific marker were measured.

Specifically, Raw 264.7 cells were dispensed into a 96-well plate at a density of 3×10³ cells/well, and were treated with RANKL to a final concentration of 100 ng/ml, and then horse chestnut extract according to Example 1 at concentrations of 1.25, 2.5, 5, and 10 μg/ml, respectively. Cells were cultured at 37° C. in a 5% CO₂ incubator for 4 days.

In addition, avicularin, quercitrin, juglanin, and afzelin which are the main ingredients of horse chestnut extract, were treated at concentrations of 2.5, 5, 10, and 25 μM, respectively, and cultured in the same manner.

After removing the media, 50 μl of extraction solution was added to each well, and pipetting was performed after reaction for 5 minutes.

50 μl of substrate solution (3,3′,5,5′-Tetramethylbenzidine) was added to each well, and reacted at 37° C. for 60 minutes. Then 50 μl of stop solution (0.5N NaOH) was added to each well, and an absorbance was measured at 405 nm.

### (2) Results

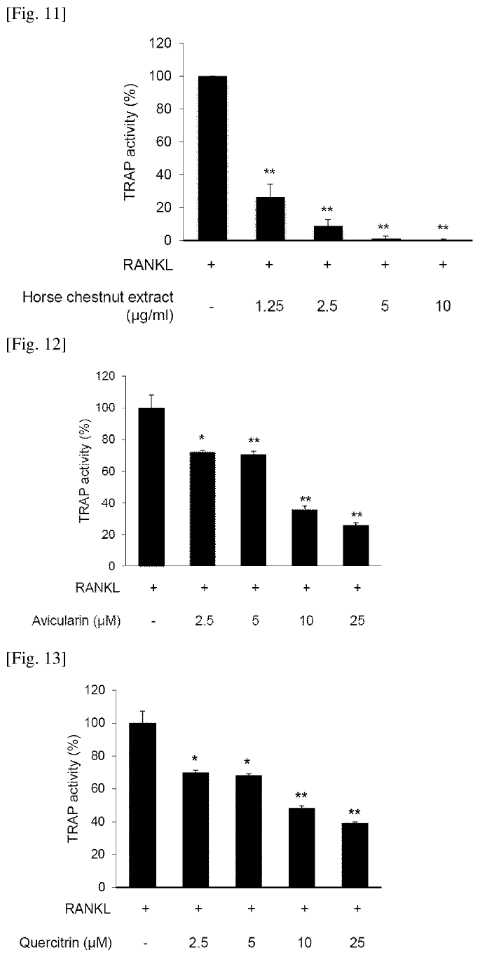

As a result, as shown in FIG. 11, horse chestnut extract according to Example 1 inhibited TRAP activity from a concentration of 1.25 μg/ml. Specifically, about 73% of activity was inhibited at 1.25 μg/ml, 91% of activity was inhibited at 2.5 μg/ml, and 98% or more was inhibited at 5 μg/ml. It was confirmed that the higher the concentration of horse chestnut extract treated, the better the TRAP inhibition effect (see FIG. 11).

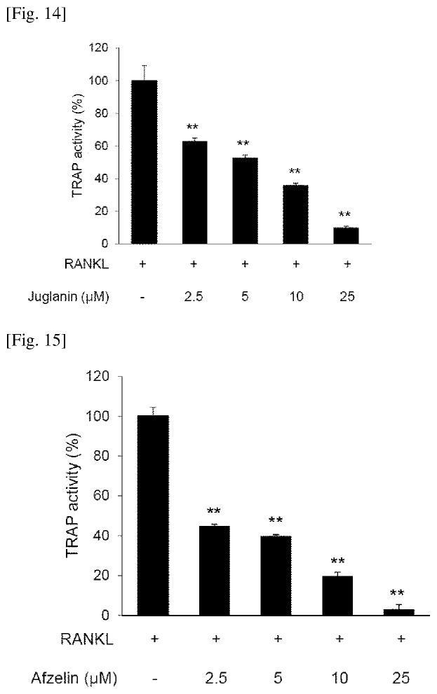

In addition, it was confirmed that avicularin, quercitrin, juglanin, and afzelin each showed inhibition of TRAP activity from 2.5 μM, but horse chestnut extract according to Example 1 showed much better TRAP inhibitory activity when compared with a single ingredient (see FIGS. 12 to 15).

Therefore, horse chestnut extract according to Example 1 of the present invention can be applied as a pharmaceutical composition for preventing or treating periodontal disease and a food composition for preventing or improving periodontal disease and can be useful for preventing, treating and/or improving periodontal disease. It has significantly fewer side effects and can be expected to prevent and/or treat periodontal disease and improve symptoms.

1. A pharmaceutical composition for preventing or treating periodontal disease comprising horse chestnut extract as effective component, wherein the horse chestnut extract comprises avicularin and juglanin as active ingredients.

2. The pharmaceutical composition of claim 1, wherein the horse chestnut extract comprises 0.1 to 4.0% by weight of avicularin and 0.1 to 3.0% by weight of juglanin as active ingredients based on the total weight of horse chestnut extract.

3. The pharmaceutical composition of claim 1, wherein the horse chestnut extract further comprises quercitrin and afzelin as active ingredients.

4. The pharmaceutical composition of claim 3, wherein the horse chestnut extract further comprises 1.0 to 7.0% by weight of quercitrin and 0.1 to 3.0% by weight of afzelin as active ingredients based on the total weight of horse chestnut extract.

5. The pharmaceutical composition of claim 1, wherein the pharmaceutical composition inhibits tartrate-resistant acid phosphatase (TRAP), an osteoclast differentiation marker.

6. The pharmaceutical composition of claim 1, wherein the horse chestnut extract is obtained by an extraction process comprising extracting horse chestnut leaves with 10% to 50% (v/v) of aqueous alcohol solution and concentrating.

7. The pharmaceutical composition of claim 1, a content of the horse chestnut extract is 10% by weight to 90% by weight based on the total weight of the pharmaceutical composition.

8. The pharmaceutical composition of claim 1, wherein the periodontal disease is at least one selected from the group consisting of gingivitis, periodontitis, alveolar pyorrhea, and peri-implantitis.

9. A food composition comprising horse chestnut extract as effective component, wherein horse chestnut extract comprises avicularin and juglanin as active ingredients.

10. The food composition of claim 9, wherein the horse chestnut extract comprises 0.1 to 4.0% by weight of avicularin and 0.1 to 3.0% by weight of juglanin as active ingredients based on the total weight of horse chestnut extract.

11. A toothpaste comprising the pharmaceutical composition for preventing or treating periodontal disease according to claim 1.

12. A gargle solution comprising the pharmaceutical composition for preventing or treating periodontal disease according to claim 1.

13. Oral spray comprising the pharmaceutical composition for preventing or treating periodontal disease according to claim 1.

14. A method for preventing or treating periodontal disease by administering a therapeutically effective amount of pharmaceutical composition comprising horse chestnut extract as effective component according to claim 1 to a subject in need of treatment.

15. (canceled)

16. (canceled)

17. A method for preventing or treating periodontal disease, comprising,
administering to a subject in need thereof the pharmaceutical composition according to claim 1.
